(19) United States
(12) Patent Application Publication
(10) Pub. No.: US 2016/0113715 A1
(43) Pub. Date: Apr. 28, 2016

(54) Probe Cover System

(71) Applicant: Kathy E. Dockett, Pine Bluff, AR (US)

(72) Inventor: Kathy E. Dockett, Pine Bluff, AR (US)

(21) Appl. No.: 14/521,749

(22) Filed: Oct. 23, 2014

(51) Int. Cl. A61B 19/02; A61B 5/01; G01K 1/08; B32B 5/18; B32B 27/06; A61B 1/227; B32B 1/08

(52) U.S. Cl. CPC A61B 19/02; A61B 1/227; A61B 5/01; B32B 1/08; B32B 5/18; B32B 27/065; G01K 1/086; B32B 2250/02; B32B 2535/00

(57) ABSTRACT

A probe cover system for improving comfort when using medical instruments with probes. The probe cover system generally includes a probe cover adapted for use on the probe of a medical instrument such as an otoscope or thermometer. The probe cover includes a wide inner end and a narrow outer end. The outer end includes a first opening and the inner end includes a second opening, with the first opening generally being narrower than the second opening. The probe cover includes a plastic outer layer and a cushioned, foam inner layer to improve the comfort of the patient while the medical instrument is in use.

### CROSS REFERENCE TO RELATED APPLICATIONS

Not applicable to this application.

### STATEMENT REGARDING FEDERALLY SPONSORED RESEARCH OR DEVELOPMENT

Not applicable to this application.

### BACKGROUND OF THE INVENTION

1. Field of the Invention

The present invention relates generally to a probe cover and more specifically it relates to a probe cover system for improving comfort when using medical instruments with probes.

2. Description of the Related Art

Any discussion of the related art throughout the specification should in no way be considered as an admission that such related art is widely known or forms part of common general knowledge in the field.

Various medical instruments include probes which come in contact with or enter various parts of a patient's body. These probes are often comprised of metal or metal alloys which can be cold to the touch. While doctors and nurses commonly attempt to warm up these probes, it is all-to-common for patients to experience discomfort from probes retaining cold properties prior to being applied to the body.

Further, probes are commonly comprised of hard materials, such as metals and the like. For probes attached to medical instruments for entering the body, such as otoscopes or thermometers, it can be uncomfortable when the probe enters the body due to the rigidity of the probe surface.

Because of the inherent problems with the related art, there is a need for a new and improved probe cover system for improving comfort when using medical instruments with probes.

### BRIEF SUMMARY OF THE INVENTION

The invention generally relates to a cushioned probe cover which includes a probe cover adapted for use on the probe of a medical instrument such as an otoscope or thermometer. The probe cover includes a wide inner end and a narrow outer end. The outer end includes a first opening and the inner end includes a second opening, with the first opening generally being narrower than the second opening. The probe cover includes a plastic outer layer and a cushioned, foam inner layer to improve the comfort of the patient while the medical instrument is in use.

There has thus been outlined, rather broadly, some of the features of the invention in order that the detailed description thereof may be better understood, and in order that the present contribution to the art may be better appreciated. There are additional features of the invention that will be described hereinafter and that will form the subject matter of the claims appended hereto. In this respect, before explaining at least one embodiment of the invention in detail, it is to be understood that the invention is not limited in its application to the details of construction or to the arrangements of the components set forth in the following description or illustrated in the drawings. The invention is capable of other embodiments and of being practiced and carried out in various ways. Also, it is to be understood that the phraseology and terminology employed herein are for the purpose of the description and should not be regarded as limiting.

### BRIEF DESCRIPTION OF THE DRAWINGS

Various other objects, features and attendant advantages of the present invention will become fully appreciated as the same becomes better understood when considered in conjunction with the accompanying drawings, in which like reference characters designate the same or similar parts throughout the several views, and wherein:

FIG. 1 is an upper perspective view of the present invention with the probe cover aligned for installation.

FIG. 2 is an upper perspective view of the present invention installed on a medical instrument.

FIG. 3 is an upper perspective view of an alternate embodiment of the present invention with the probe cover aligned for installation.

FIG. 4 is an upper perspective view of an alternate embodiment of the present invention installed on a medical instrument.

FIG. 5 is an upper perspective view of the present invention.

FIG. 6 is a rear view of the present invention.

FIG. 7 is a frontal view of the present invention.

FIG. 8 is an upper perspective view of an alternate embodiment of the present invention.

FIG. 9 is a rear view of an alternate embodiment of the present invention.

FIG. 10 is a frontal view of an alternate embodiment of the present invention.

FIG. 11 is a sectional view taken along line 11-11 of FIG. 6.

FIG. 12 is a sectional view taken along line 12-12 of FIG. 9.

### DETAILED DESCRIPTION OF THE INVENTION

### A. Overview

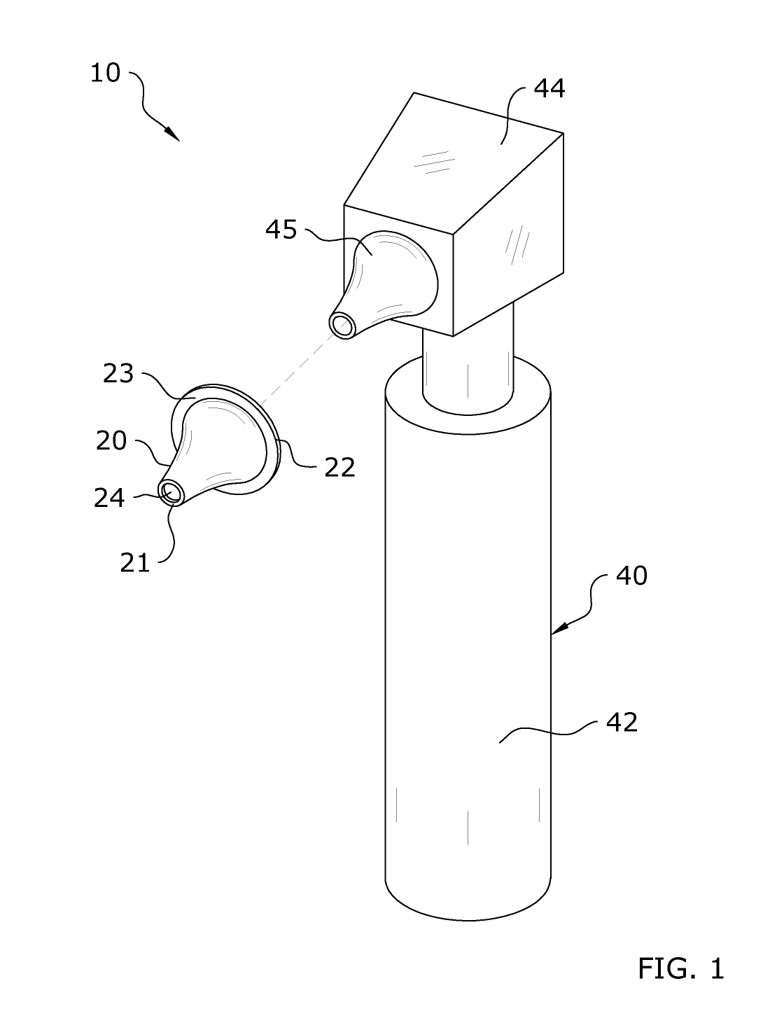

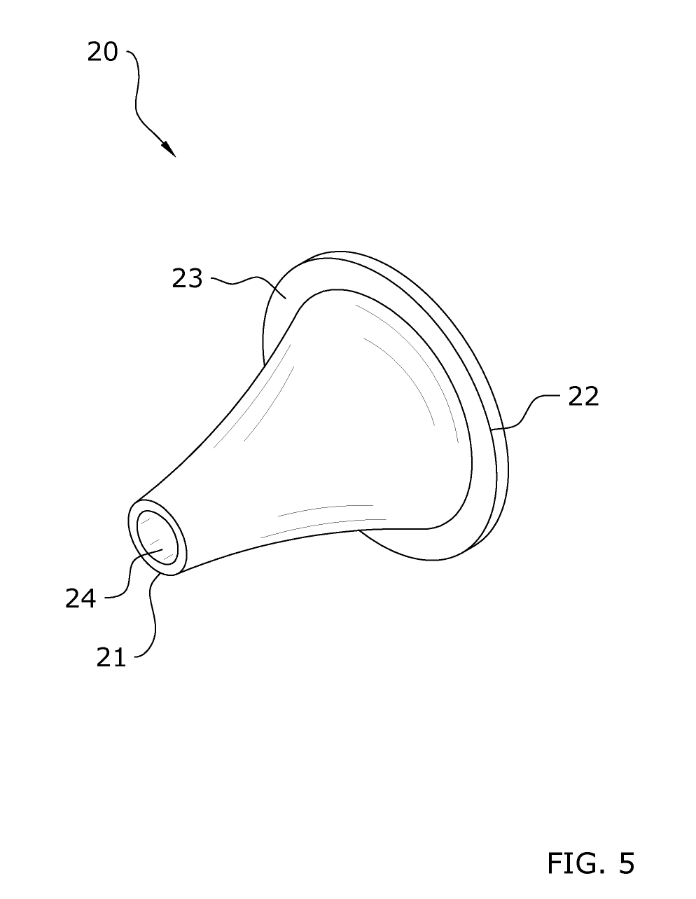

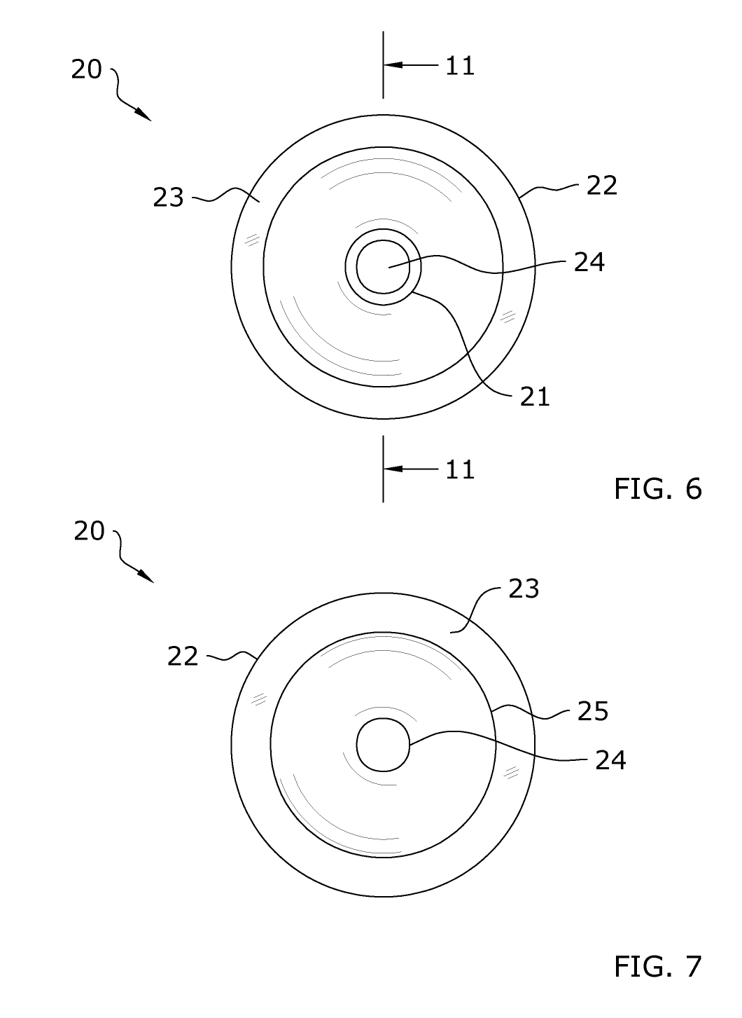

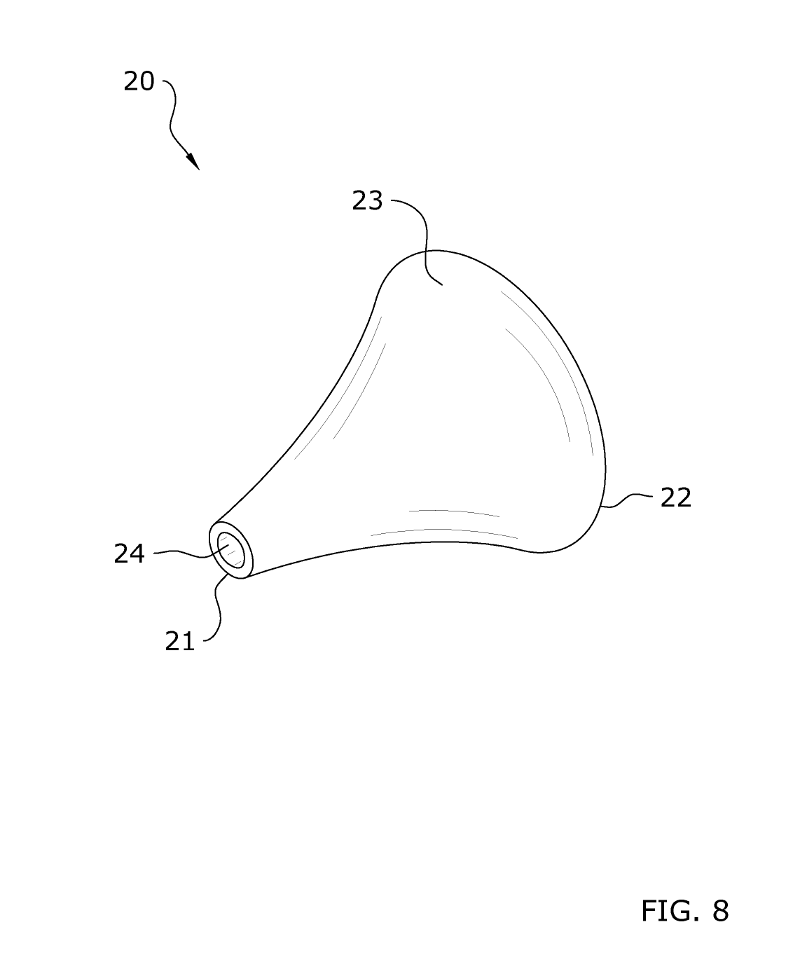

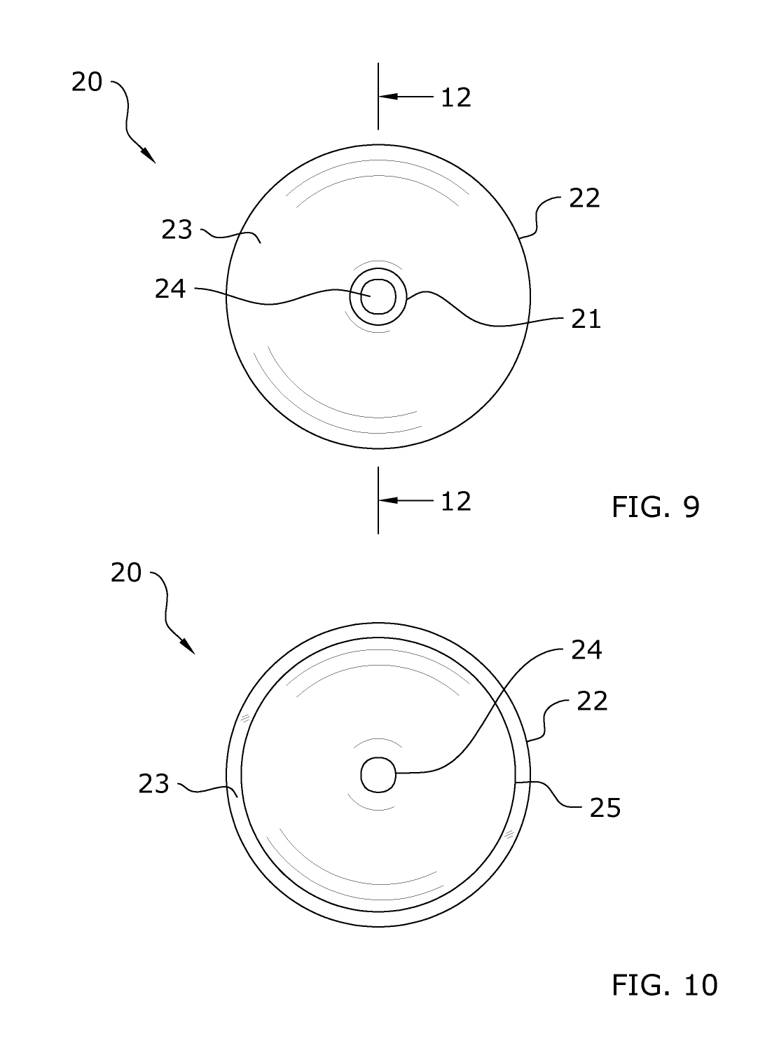

Turning now descriptively to the drawings, in which similar reference characters denote similar elements throughout the several views, FIGS. 1 through 12 illustrate a probe cover system 10, which comprises a probe cover 20 adapted for use on the probe 45 of a medical instrument 40 such as an otoscope or thermometer. The probe cover 20 includes a wide inner end 22 and a narrow outer end 21. The outer end 21 includes a first opening 24 and the inner end 22 includes a second opening 25, with the first opening 24 generally being narrower than the second opening 25. The probe cover 20 includes a plastic outer layer 30 and a cushioned, foam inner layer 32 to improve the comfort of the patient while the medical instrument 40 is in use.

### B. Probe Cover

As shown throughout the figures, the present invention utilizes a probe cover 20 which greatly increases the comfort when utilizing a medical instrument 40 with a probe 45 adapted to enter an area of the body, such as an ear. It should be appreciated that the shape, structure, configuration, and size of the probe cover 20 may vary widely in different embodiments of the present invention. For example, the probe cover 20 may be sized differently for different patients. Probe covers 20 for children will be significantly smaller than probe covers 20 for adults.

As shown in the figures, the probe cover 20 will generally include an outer end 21 and an inner end 22. The outer end 21 comprises the portion of the probe cover 20 which enters the body part, such as an ear. The inner end 22 is secured (generally frictionally) onto the probe 45 of the medical instrument 40.

The outer end 21 of the probe cover 20 includes a first opening 24 and the inner end 22 of the probe cover 20 includes a second opening 25. The medical instrument's 40 functionality is performed through the first opening 24. The second opening 25 is utilized to secure the probe cover 20 around the probe 45 as shown in the figures. Preferably, the first opening 24 will be considerably smaller than the second opening 25, though other configurations may be utilized.

As shown throughout the figures, the probe cover 20 will generally comprise a cone structure. Thus, the inner end 22 of the probe cover 20 will preferably be wider than the outer end 21 of the probe cover 20 as shown throughout the figures to match the cross-section of most typical probes 45. This may vary in different embodiments, however.

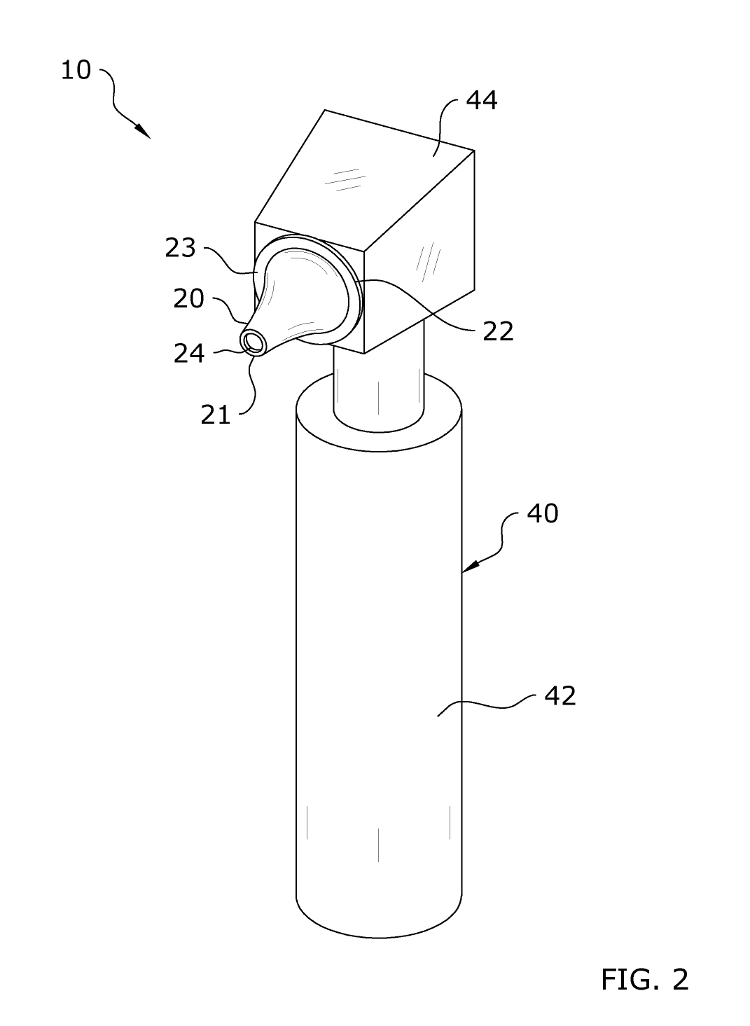

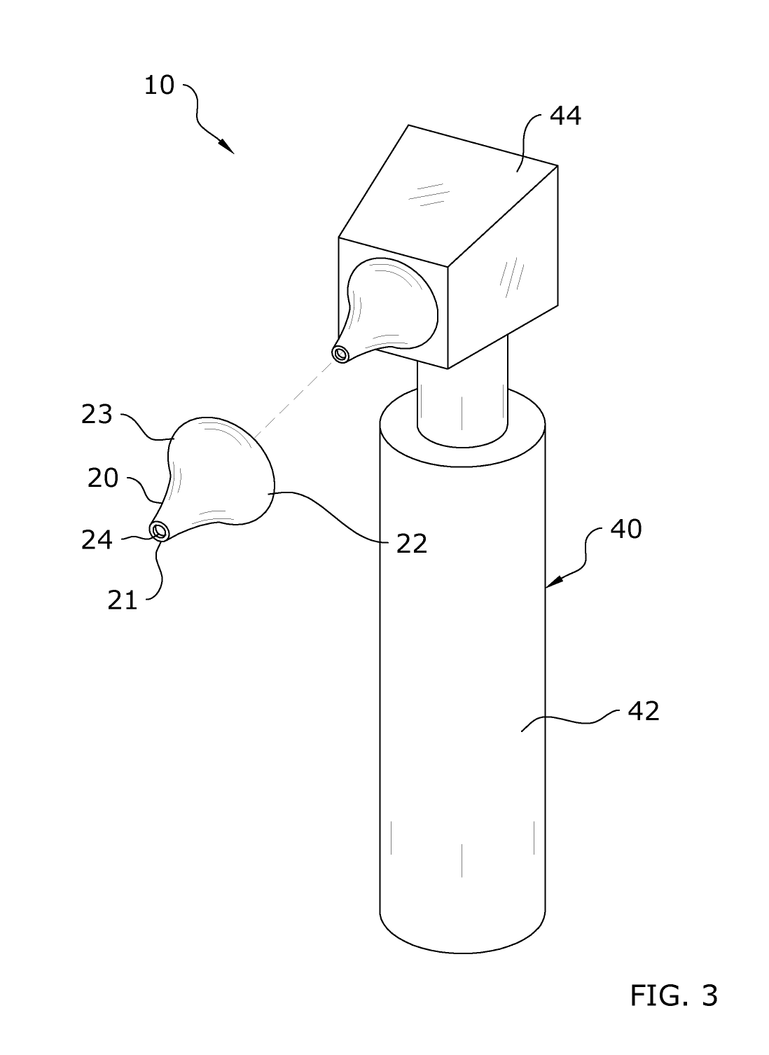

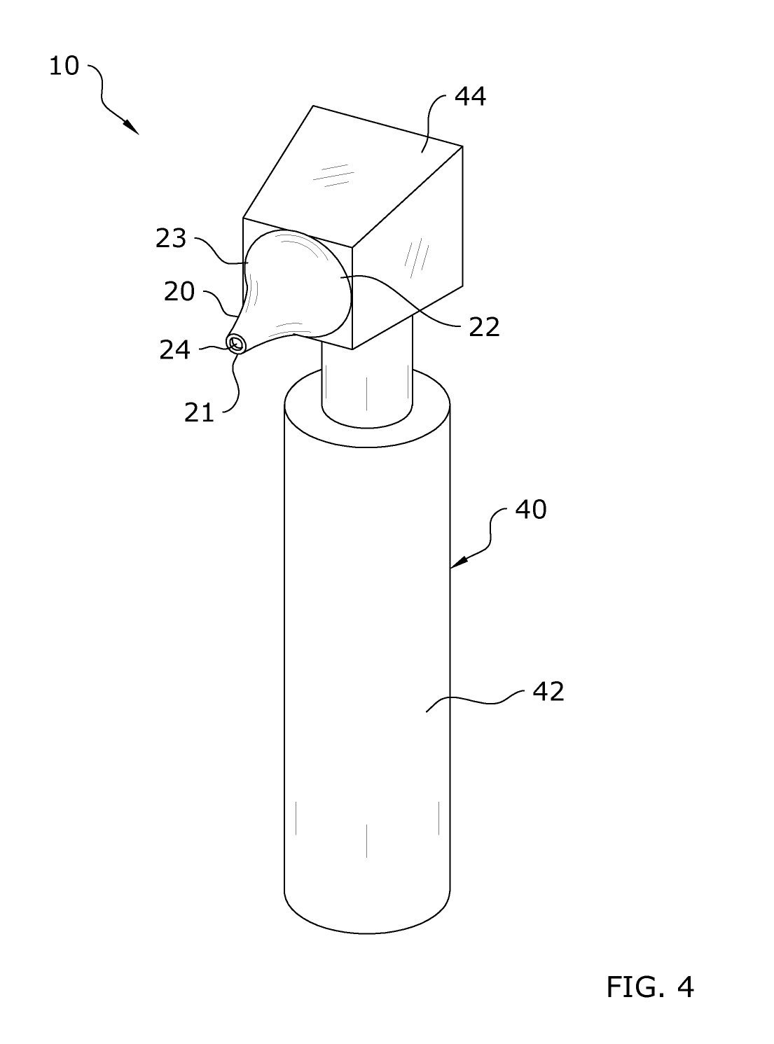

The figures illustrate two exemplary embodiments of the present invention. FIGS. 1-2 illustrate a first embodiment adapted for use with a medical instrument 40 comprised of a thermometer. FIGS. 3-4 illustrate a second embodiment adapted for use with a medical instrument 40 comprised of an otoscope.

The first embodiment shown in FIG. 1 includes a slightly wider first opening 24 than the second embodiment shown in FIG. 2. Further, the second embodiment shown in FIG. 2 includes an optional base 23 which surrounds the inner end 22 of the probe cover. This base 23 aids with securing the probe cover 20 onto the probe 45 of the medical instrument 40.

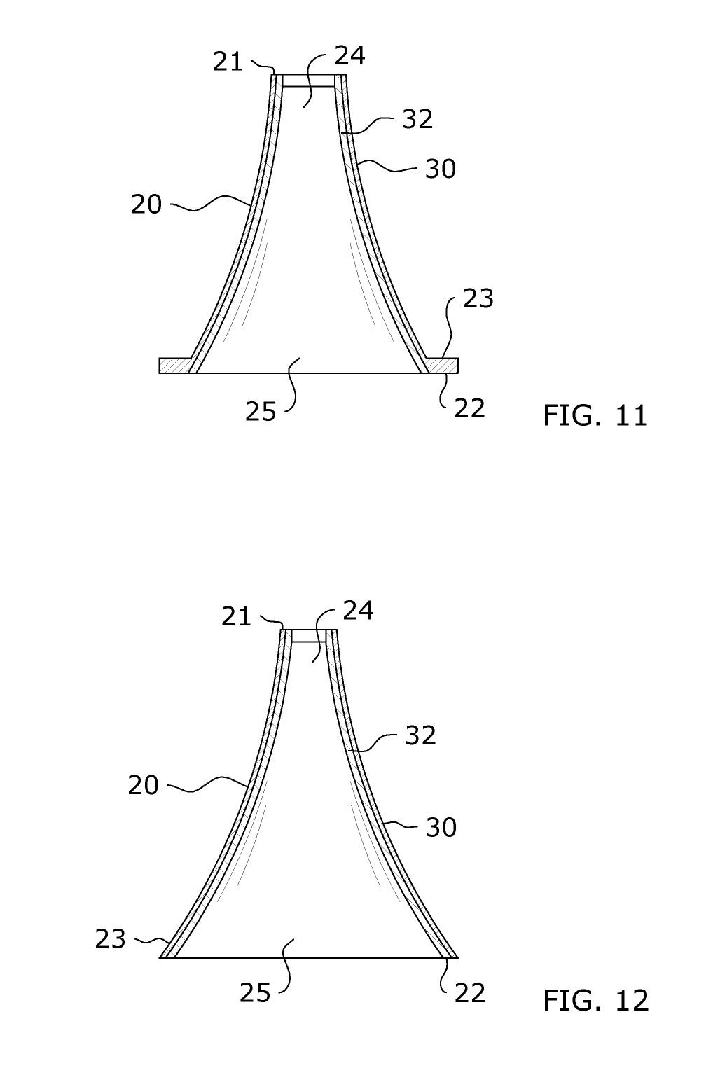

As best shown in FIGS. 11-12, the probe cover 20 comprises an outer layer 30 of a first material and an inner layer 32 of a second material. The outer layer 30 of the probe cover 20 will generally comprise a soft plastic material which is comfortable for the patient. The inner layer 32 of the probe cover 20 generally comprises a foam cushion made of any foam-type material (such as cotton or the like) to create a cushion between the body of the patient and the hard underlying probe 45. This greatly improves comfort of the patient by providing a soft surface and eliminating the cold feelings associated with the metallic probes 44 commonly used on such medical instruments 40.

The probe cover 20 may be secured to the probe 45 of the medical instrument 40 through various methods. Preferably, the probe cover 20 will fit tightly enough onto the probe 45 that a frictional engagement is achieved that prevents the probe cover 20 from being dislodged during use but allows it to be easily pulled off the probe 45 when desired. In alternate embodiments, adhesives, tapes, clamps, and the like could be utilized to secure the probe cover 20 to the probe 45.

### C. Medical Instrument

The present invention is adapted for use with any medical instrument 40 which includes a portion, such as a probe 45, which enters or contacts the body of a patient. The figures illustrate exemplary embodiments in which the medical instrument 40 comprises either an otoscope or a thermometer. It should be appreciated that these are merely exemplary uses, and that the present invention could be utilized on a wide range of medical instruments 40.

Generally speaking, the medical instrument 40 will include a handle 42, a head 44 extending from the handle 42, and a probe 45 extending from the head 44. As previously mentioned, any number of these components could be omitted from certain medical instruments 40 used with the present invention. For example, the medical instrument 40 need not include a handle 42 in every embodiment of the present invention.

### D. Operation of Preferred Embodiment

In use, the probe cover 20 is fitted onto the probe 45 of the medical instrument 40 as shown in FIGS. 1-4. The second opening 25 of the inner end 22 is fitted over the probe 45 such that the probe 45 is covered while still allowing signal access through the first opening 24 at the outer end 21. The medical instrument 40 is then ready for use by inserting the probe 45 into the relevant area of the body, such as an ear. After use is completed, the probe cover 20 may be removed by pulling off from the probe 45. Generally, the outer surface of the probe cover 20 will be grasped between the fingers and then pulled away from the probe 45, allowing the probe cover 20 to easily slide off the probe 45. The probe cover 20 may then be discarded or stored for further usage.

Unless otherwise defined, all technical and scientific terms used herein have the same meaning as commonly understood by one of ordinary skill in the art to which this invention belongs. Although methods and materials similar to or equivalent to those described herein can be used in the practice or testing of the present invention, suitable methods and materials are described above. All publications, patent applications, patents, and other references mentioned herein are incorporated by reference in their entirety to the extent allowed by applicable law and regulations. The present invention may be embodied in other specific forms without departing from the spirit or essential attributes thereof, and it is therefore desired that the present embodiment be considered in all respects as illustrative and not restrictive. Any headings utilized within the description are for convenience only and have no legal or limiting effect.

The invention claimed is:

1. A probe cover, comprising:
an inner end adapted to be secured to a medical instrument;
an outer end adapted to contact a patient;
an inner layer comprised of a first material; and
an outer layer comprised of a second material.

2. The probe cover of claim 1, wherein said first material comprises a cushion material.

3. The probe cover of claim 2, wherein said first material comprises a foam.

4. The probe cover of claim 2, wherein said second material comprises a plastic material.

5. The probe cover of claim 1, wherein said probe cover is frictionally secured to said medical instrument.

6. The probe cover of claim 1, wherein said inner end is wider than said outer end.

7. The probe cover of claim 1, wherein said probe cover comprises a cone structure.

8. The probe cover of claim 1, wherein said outer end includes a first opening and wherein said inner end includes a second opening.

9. The probe cover of claim 1, wherein said first opening is narrower than said second opening.

10. A probe cover system, comprising:
a medical instrument; and
a probe cover, wherein said probe cover is adapted to be removably secured to said medical instrument, said probe cover comprising;
an inner end adapted to be secured to said medical instrument;
an outer end adapted to contact a patient;
an inner layer comprised of a first material; and
an outer layer comprised of a second material.

11. The probe cover system of claim 11, wherein said first material comprises a cushion material.

12. The probe cover system of claim 12, wherein said first material comprises a foam.

13. The probe cover system of claim 12, wherein said second material comprises a plastic material.

14. The probe cover system of claim 11, wherein said probe cover is frictionally secured to said medical instrument.

15. The probe cover system of claim 11, wherein said inner end is wider than said outer end.

16. The probe cover system of claim 11, wherein said probe cover comprises a cone structure.

17. The probe cover system of claim 11, wherein said medical instrument is comprised of an otoscope.

18. The probe cover system of claim 11, wherein said medical instrument is comprised of a thermometer.

19. The probe cover system of claim 11, wherein said probe cover includes a base at said inner end.

20. A probe cover system, comprising:
a medical instrument, wherein said medical instrument includes a probe; and
a probe cover for covering said probe, said probe cover comprising;
an inner end adapted to be secured to said medical instrument;
an outer end adapted to contact a patient, wherein said outer end is narrower than said inner end;
a first opening on said outer end;
a second opening on said inner end, wherein said first opening is narrower than said second opening;
an inner layer comprised of a foam material; and
an outer layer comprised of a plastic material.
